(19) United States
(12) Patent Application Publication
(10) Pub. No.: US 2008/0193993 A1
(43) Pub. Date: Aug. 14, 2008

(54) Method and Plant for Producing Biogas from Bio-Organic Residual Matters

(76) Inventor: Gunter Schulze, Dresden (DE)

Correspondence Address: BROOKS KUSHMAN P.C., 1000 TOWN CENTER, TWENTY-SECOND FLOOR, SOUTHFIELD, MI, 48075, US

(21) Appl. No.: 11/912,214

(22) PCT Filed: Mar. 23, 2006
(86) PCT No.: PCT/DE2006/000516

§ 371 Date: Oct. 22, 2007

(30) Foreign Application Priority Data
Apr. 22, 2005 (DE) 10 2005 019 445.1

(51) Int. Cl. C12P 5/02

(52) U.S. Cl. 435/167; 48/61

(57) ABSTRACT

The invention relates to a plant and method for producing a biogas from different organic waste materials from household, agriculture, forestry, industry and commerce sectors (bioorganic residues) by anaerobic alkaline sludge digestion. The aim of said invention, in particular, is to substantially reduce a production and energy costs and, thereby a manpower cost for preparing bio-organic even difficult-to-degrade residues for converting a large quantity of substance amount into a methane-containing gas used as an energy source. For this purpose, the inventive method consists in using at least two more or less horizontal underground cavities, which previously were used as coal mines, in the form of digestion tanks, in connecting all mine cavities into one or several spots by defined gas discharging drillings in such a way that all drillings enter a gas collecting container arranged at the highest point of the mine, in introducing a large quantity of non-reduced bio-organic residues into the digestion tanks, wherein a slug digestion is carried out by long-term reactions without additional heating at temperatures ranging from 5 to 70° C.

The invention relates to a plant for producing biogas from different biomass from households, agriculture, forestry, industry, and commerce sectors (biomass) by anaerobic alkaline sludge digestion by means of different strains of methane bacteria with a digester and a feed pipe for the biomass.

The invention also relates to a method for producing biogas from biomass, in which method different biomasses are introduced into at least one naturally existing digester and there converted into methane-containing biogas by means of different strains of methane bacteria according to the principle of anaerobic alkaline sludge digestion.

Methane-containing gas is for example also produced in the excavations of hard coal mines (mine gas). Mine gas, too, consists of the two main components methane and carbon dioxide, just like biogas. In hard coal mines, the mine gas issues from the seams due to their loosening and the reduction of the pressure on them. As described in the introductory description of the German patent application publication 1 758 628, the mine gas is extracted directly from the seams by means of drillings during the exploitation, in order to produce sufficient quantities of mine gas and at the same time to avoid potentially explosive mixtures of gas and air. Although, due to the air contained in the shafts of the exploited hard coal mine, the produced gas is a mixture of methane and air, the methane content, being 80%, is high enough for technical utilisation. However, the mixture contains considerable quantities of air when disused shafts are closed and the mine gas contained there is extracted.

Utilisation of a mine shaft for a waste water sewage plant is known from the German patent application publication 35 38 183. However, this only makes use of the vertical shafts for waste water treatment, but no biogas is produced.

For a long time, it has been part of the Prior Art to decompose sewage sludge from the sedimentation installations of big municipal sewage plants by means of methane bacteria in closed digester facilities at a temperature of 28 to 42° C. (mesophilic range) within 10 to 21 days in the first digestion stage, and to convert part of the bio-organic substance into biogas. In some rare cases, the biological process is carried out at a temperature of 42 to 60° C. (termophilic range). Subsequently to the first digestion stage, a second digestion stage is conducted to further decompose the bio-organic substance in open, unheated secondary digester reservoirs within a residence time of up to 100 days. A disadvantage of the known methods is the enormous energy demand for the heating of the bio-organic substance to reach the chosen digester temperature. This is also associated to high carbon dioxide emissions. Of late, known solutions propose adding liquid manure from livestock husbandry and/or other bio-organic residues from households and industry sectors to the sewage sludge. The biogas produced from these substances is mainly burned in boiler plants and the steam thereby produced is used to heat the feed material in closed digester plants. Surplus biogas is mainly used for power generation in block heat and power plants in the warm season. In winter, the sewage sludge from the sewage plants and the added bio-organic substances have to be heated from an average 5° C. to about 33° C., this process using up almost all the produced biogas.

It is also known that bio-organic substances composed of dried fresh sewage sludge and/or digestion sludge is mixed with other bio-organic residues and composted. Subsequently, it is used in agriculture or for the recultivation of desolate areas. DE-OS 4003487 describes a method for the stabilisation of sludge introduced in a digester, in which an aerobic/anaerobic treatment is carried out during a preliminary stage. The disadvantage of this method is that with an aerobic preliminary treatment, no biogas is produced, but only carbon dioxide. According to the German patent application publication 1 758 628 a method is described for the production of mine gas in a previously partly exploited subterranean hard coal deposit by closing the entry shafts and conducting the mine gas from the mining points to the surface. The Austrian patent 361 015 describes a method for the production of biogas and a plant in the form of an array of several aboveground fermentation and sedimentation boilers for aerobically pre-treated bio-organic waste matter. The patent application publication of the European patent 1 488 855 describes a method and plant for the production of biogas from bio-organic residues, in which the bio-organic residues are milled and pressed, giving off water, before being partly biologically degraded. The pressing has to be done in such a way that the loss of weight of the bio-organic residues due to the yielded water is at least 50%. All these known solution have disadvantages such as high technical effort, with extremely high operational costs and limited utilisation possibilities of the produced gas. A complete abstract of the methods and plants of the Prior Art can be found in the special reference book “Anaerobe alkalische Schlammfaulung” (“Anaerobic alkaline sludge digestion”) by H. Roediger, M. Roediger and H. Kapp, published by Oldenbourg-Verlag Munich Vienna (4^{th} edition, volume 1990). It is also known that household and industrial residues are deposited in landfill sites without preliminary treatment. Composting starts due to the atmospheric oxygen, the greater part of the bio-organic substance of these residual matters being aerobically converted by means of the process heat into carbon dioxide, which is harmful to the environment. Only when further great quantities of residues are deposited, access of atmospheric oxygen is restricted and aerobe micro-organisms die off. After some time, methane bacteria take over the decomposition of more bio-organic substances, producing landfill gas. Small quantities of landfill gas or gas with a small methane content are flared. Only greater quantities can be used for power generation in block heat and power plants.

Further disadvantages of such known solutions, which are mainly used for the disposal of bio-organic residues, are an enormous energy consumption needed for heating-up the bio-organic residues to the determined digester temperature and for compensating the unavoidable loss from radiation, as well as the production of considerable quantities of carbon dioxide due to the burning of biogas or fossil fuels. If bio-organic residual matter from households and industrial sectors is added to municipal sewage sludge or liquid manure from livestock husbandry, and the mixture is subjected to anaerobic alkaline sludge digestion in digester plants, a mechanical degradation—as for example described in EP 1 488 855 A1—is necessary in order to enable the bacterial degradation within the short digestion time and the extraction of the matter by sludge pumps. Prior to the degradation of the bio-organic matter to be added, an expensive process of sorting out and disposing of contaminants (stones, glass, metal and plastic) is required in order to avoid damages to the plants. These processes are very costly in financial and technical terms. The biogas with a high methane content which is produced in open secondary digester reservoirs is emitted unutilised into the atmosphere and encourages the greenhouse effect. Screened trash from municipal sewage plants, composed mainly of polluted paper waste and textiles, has to be deposited in landfill sites or burned. Utilisation has not been possible so far. The main disadvantage of the composting of bio-organic residues is the fact that the decomposed bio-organic substance is converted 100% into carbon dioxide, resulting in a considerable environmental pollution with this greenhouse gas. In addition to the technical and ecological disadvantages, the known methods for the preliminary treatment of bio-organic residual matter also cause considerable costs.

In a method for producing bio-organic matter, described in the German patent application publication 100 62 030, abandoned cavities with saliniferous walls in disused salt mines are used for the fermentation instead of closed or open fermentation tanks, in order to enable the production of greater quantities of bio-organic matter without the construction of elaborate and non-corrosive production facilities. However, only such processes can be used in which halophilic organisms are used for the conversion, that means organisms for the cultivation of which salt is needed in great quantities and which can work only under such conditions. The production of biogas is not part of the method presented.

The objective of the invention is therefore to present a plant and a method for producing biogas from biomass which considerably reduces the production and energy costs and, moreover, the manpower costs for preparing bio-organic, difficult-to-degrade residues for converting a large quantity of substance into a methane-containing gas usable as an energy source. Furthermore, the demands of global climate protection are to be met and the greenhouse effect of carbon dioxide caused by the traditional burning of the methane-containing or fossil fuels is to be avoided.

This objective is achieved by a plant with the characteristics according to claim 1 and a method according to claim 10. The layout of the plant and the method are indicated in the sub-claims.

For this form of producing biogas from bio-organic residues carried out by long-term reactions according to the principle of anaerobic alkaline sludge digestion by means of different strains of methane bacteria, such disused hard coal mines are especially suitable because the investment costs for the construction of closed digestion facilities are eliminated. For this purpose it is necessary to include the existing cavities within the mine, the galleries, levels and drifts in the solution according to the invention.

An especially positive effect of the invention is the fact that geothermal power can be used as energy source for creating a certain temperature level without additional heating to provide for ideal living and reaction conditions for the methane bacteria.

Since methane bacteria are very adaptable and have different strains, biomasses in the temperature range of 5° C. to 70° C. are converted into biogas, in the cryophilic range (below 10° C.), the mild range (between 10° C. and 28° C.), the mesophilic range (between 28° C. and 42° C.) and the thermophilic temperature range (between 42° C. and 70° C.). Only at a temperature above 70° C. the methane bacteria die off. Therefore, cavities in which a temperature within this temperature range can be maintained throughout the long-term reaction, taking into account the self-heating of the bio-organic residuals, are usable as digesters.

The plant according to the invention as well as the method enables the following triple utilisation of renewable energies:
a) Energy recovery from the small quantities of mine gas accumulated, avoiding the greenhouse effect;
b) Heating-up of the introduced bio-organic residuals by geothermal power, saving a lot of heating costs and keeping carbon dioxide-containing greenhouse gases from being emitted to the environment and
c) Production of biogas by methane bacteria carried out in long-term reactions of up to 20 years, almost completely decomposing even persisting residual matters such as hedge and tree clippings. According to the methods known so far with a residence time of 10 to 21 days, these cellulose residues remained in their original state.

The produced biogas can be fed into known gas utilisation facilities such as block heat and power plants and/or high-temperature fuel cells for power generation. Furthermore, the plant, which works without hazard to the environment, can be combined with known kinds of mine gas production and can be connected to facilities for the economic utilisation of the produced gas, especially for electrical power generation.

Very advantageous synergy effects can be achieved if such hard coal mines are refitted for the plant according to the invention from which mine gas has already been extracted and used for energy recovery, but which were no longer working efficiently due to insufficient mine gas quantities. The combination of mine and biogas production in disused subterranean mines is an especially positive effect of the invention. Since the mine has already been used for mine gas production and all cavities are connected via the ventilation system, no further drillings between the galleries, shafts and blind shafts were necessary.

Already existing plant parts and facilities for the utilisation of the mine gas, such as aspirators and block heat and power plants, can again be operated and used for the biogas production according to the invention with minimum retrofitting efforts. The combination of mine gas and biogas production leads to an even higher gas yield. Even small remaining quantities of mine gas can be used economically together with the produced biogas and are therefore not emitted into the atmosphere. Thus, an integration of a gas production from unexploited seams into the plant and the method according to the invention is also advantageous for the production of biogas which can be used for energy recovery.

Furthermore, the invention proposes that such subterranean mines which are projected for mine gas utilisation in the future are combined with biogas production and a combined utilisation from the outset, so as to have a maximum energy recovery effect.

Of course it is also possible, according to the invention, to subsequently use a combination with anaerobic alkaline sludge digestion in mines with big quantities of mine gas and extraction and utilisation already carried out.

The gas production plant, consisting in a disused subterranean mine with numerous complex cavities left over from the mining activities, such as galleries, levels and mine excavations, uses at least two horizontal galleries and/or levels as well as blind shafts as digesters. These are connected at one or several points by defined drillings, which lead off gas, in such a way that these gas-discharging drillings all open out at the gas collection reservoir, which is situated at the highest point of the mine. This way, the possibility of dead zones in the mine is ruled out. The diameter of the drillings to be done depends on the quantities of gas produced. Drillings at the bottom level are considerably smaller in diameter than those near the surface. On the other hand they must not be obstructed by intruding residues.

Since blind shafts do not have any connection to the surface, they can be used as gas collection shafts, for which purpose they are equipped with a supplementary drilling to the gas utilisation station. Blind shafts which are not intended for this use must also present such a drilling in order to include them in the leading-off of the gas and to lead the methane-containing gases entering into them to the gas utilisation station.

If several mines are included in a compound mine with a void volume of up to 10 million m³, the connecting galleries must be closed up in order to better control the mine and biogas production. Only when all cavities are entirely filled with bio-organic residues, other mines of the compound mine may be included in the solution according to the invention.

In bigger mines or a compound mine, a gasometer is proposed for the intermediate storage of the greater quantities of accumulated gas, the gas which is not utilised immediately after production being conducted from the gas collection reservoir within the mine into the gasometer via feed pipes at a small excess pressure of 20-50 millibar. Furthermore, inactive, already flooded mines can also be used according to the invention, from which the flood water can still be extracted from the cavities without great technical efforts.

The biogas produced in the biological process and stored in the gas collection reservoir or in the intermediate gas store respectively can either be mixed with the methane gas, which in case of the possible combination with mine gas production is extracted after removal of the carbon dioxide, or it can be fed directly into the natural gas networks, or it can be fed separately via connecting pipes into known gas utilisation facilities such as block heat and power plants and/or high-temperature fuel cells for power generation. In case of very high gas accumulation in compound mines, the carbon dioxide can also be separated from the gas mixture by the pressure or the membrane method, liquefied and subjected to utilisation. For example, carbon dioxide is very suitable as an effective fire extinguisher.

Besides the refitting of the hard coal mine as described above, the method for producing biogas according to the invention also proposes to introduce the bio-organic residues without prior degradation. In a cavity used as digester near the bottom level of the disused hard coal mine, the undegraded bio-organic matter is also converted into methane-containing biogas during a long-term reaction.

In order to initiate the biogas production immediately, an advantageous design of the invention proposes to bring the biomasses in contact with seed sludge during the stage of introduction of the matter. It is also advantageous to mix the introduced bio-organic residues with the digestion sludge by pressing in natural or biogas, in order to activate the methane bacteria and more quickly produce biogas. The adding of seed sludge can be dispensed with if a longer start-up process is acceptable. It is known from experience in mine gas production that the accumulated mine gas is extracted before utilisation. In case of a malfunction of the plant, a sudden increased gas accumulation has to be flared by means of a gas flare.

In the following, the invention will be explained in detail on the basis of a realisation example. The respective drawings show

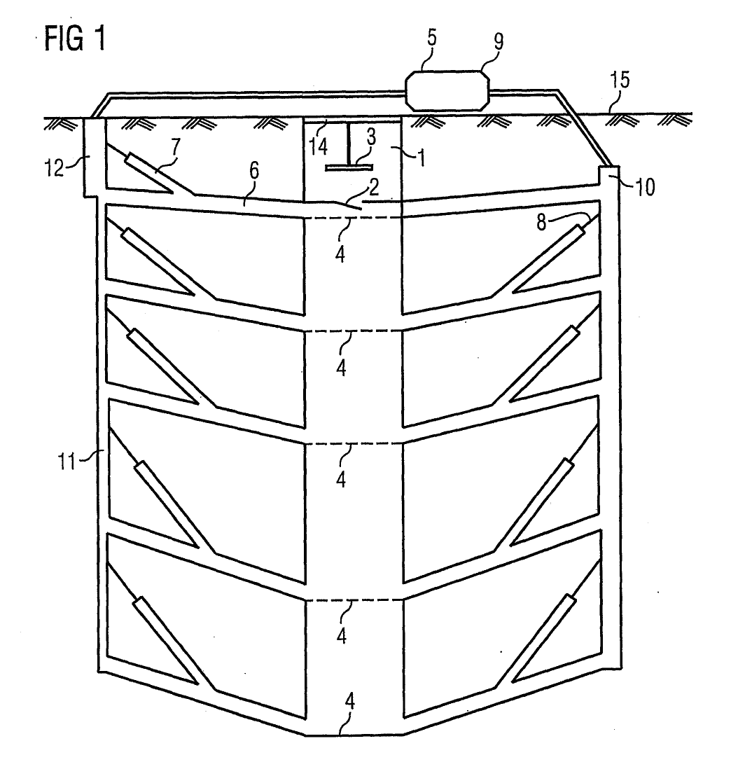

FIG. 1 a vertical section of a plant according to the invention in a schematic representation and

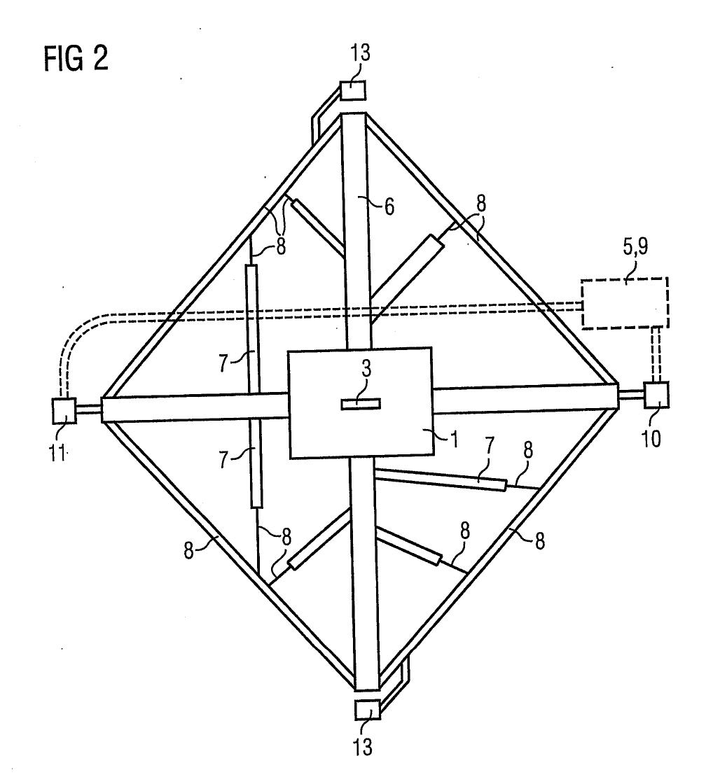

FIG. 2 a horizontal section of this plant, also in a schematic representation.

The example for utilisation according to the invention described below is a disused, not yet flooded hard coal mine with a depth of approximately 400 m, a very high mine gas accumulation and a mine volume of approximately one million cubic meters. The chosen mine was classified very dangerous during the hard coal mining, because mine gas continuously penetrated into the mining area from the coal seams and had to be removed by the ventilation system. Utilisation of these mine gases, which still issue from the seams in decreasing quantities after the closing of the mine, is proposed for the method according to the invention and completely integrated in the entire gas production. Since the accumulation of mine gas has lessened over the years, the previous mine gas utilisation will be combined with the solution according to the invention, in order to produce a mixture of mine and biogas. So far, the mine gas has gone into the atmosphere uncontrolled and caused the known greenhouse effect, which is 20 times stronger than that of carbon dioxide.

Although there is little content of mine water, all possible issues are sealed up before the bio-organic matter is introduced.

Then the points in all shafts 11, 12, galleries and blind shafts 10, levels 6 and drifts 7 are determined, at which biogas and mine gas are to be lead off. The diameter of the lead-off drillings 8 which will lead to the cavities at the determined points is approximately 20 cm. Existing ventilation drillings and ventilation shafts 11, 12 can also be used for the leading-off of the gas.

The connections between the separate galleries, shafts, levels 6 and/or drifts 7 are installed in such a way that dead zones within the mine, that means zones not included in the leading-off of the gas, are avoided. At the end of the roughly horizontal levels 6 and drifts 7 in the mine, the highest point for the leading-off of the gas is given by the rising level. At these points the determined drillings 8 to a higher level cavity are done in order to ensure the leading-off of the gas. The same method applies to the connection of all cavities up to the gas collection reservoir 9 near the surface. At the high point of a vertical blind shaft 10 a drilling 8 to a nearby cavity is done in order to also lead off the gas accumulated there. At the surface of the mine, all openings not intended for the extraction of the gas and for, the introduction of the bio-organic residues are hermetically sealed. The chosen mine has three ventilation shafts 11, 12. In the represented realisation example, two ventilation shafts 13 are sealed up and at the top part a connection from each of the shafts to a nearby level or other cavity is done. The third ventilation shaft 12 is developed as gas lead-off into a gas collection reservoir 9 and is used for the continuous extraction and utilisation of the accumulated mine and biogas.

The bio-organic residues to be introduced into the mine at about 300 tons per day can come from households as well as from agriculture, municipal or forestry industries and commerce sectors. It can for example consist of municipal sewage sludge, liquid manure from livestock husbandry, greenery, grass clippings, hedge and tree clippings, spoilt food and residues from butcheries, dairies and breweries. These bio-organic residues constitute an ideal mixture for the production of biogas. Preliminary degradation of the bio-organic matter is not necessary for the method according to the invention, because the conditions given in the mine ensure a liquefaction of the bio-organic material by the long-term reaction.

For the introduction of the bio-organic residues a mixing and stacking vessel 1 is installed in the upper 5 to 10 m of an existing entrance shaft, and an opening 2 is arranged in this vessel, controlled by a pneumatically driven slide for the introduction of the bio-organic residues into the mine and for closing the entrance shaft. Furthermore, the mixing and stacking vessel 1 is equipped with a stirring device 3. The vessel also serves as insulation during the cold season and was for this reason equipped with a cover 14 approximately at surface level 15. When all preparatory work for the utilisation of the mine according to the invention is finished, the bio-organic residues are delivered by container trucks and filled into the mixing and stacking vessel 1, simultaneously adding seed sludge from the municipal sewage plant of a nearby city. Then the content of the vessel is stirred by means of the stirring device 3 and subsequently introduced into the mine by opening the slide.

Approximately 100 cubic meters of the seed sludge are added during the introduction process. This sludge is pre-treated sewage sludge from a closed digestion tower of a municipal sewage plant, which will initiate and accelerate the production of biogas in the digester.

The temperature inside the mine used for the invention is constantly 20° C. at the bottom level 4, making this level 4 usable for the biogas production method, taking into account the self-heating of the substances to be converted. The bio-organic residues introduced into the cavities are tempered without additional energy demand by the limitlessly available geothermal power as well as by self-heating due to the anaerobic biological decomposition.

Within approximately one month, the methane content of the mine and biogas mixture rises to 45%, so that energy recovery of the produced gas in a block heat and power plant 5 is already possible after this time. With the given quantities of introduced matter, about 17,000 m³/day of biogas can be produced in this mine in addition to the mine gas, which, mixed with the mine gas, is extracted from the mine and converted into electrical energy in the already linked-up block heat and power plant 5. Due to the greater accumulation of gas the linked-up block heat and power plant 5 is equipped with another four modules with a capacity of 400 to 500 kW for each engine.

For this example, the avoided heat losses as well as the quantities of carbon dioxide kept out of the environment are hereafter calculated in comparison to the known methods in closed and heated digesters. The heat demand for sludge digestion of 300 t of bio-organic matter/day including heat losses is known to be

$$\frac{300 , 000 kg \times 4.2 kJ \text{/} kg \text{/} ° C . \times 35 ° C .}{3 , 600 kJ \text{/} kWh} = 12 , 250 kWh \text{/} d .$$

At a 65% methane content of the biogas, 12,250 kWh per day correspond to about 5,952 m³ of biogas per day, which is saved by the solution according to the invention. The greenhouse gas carbon dioxide, with an amount of 4,117.6 kg per day, which corresponds to approx. 1,503 t per year, does not get into the atmosphere because geothermal power is used.

If only 100 mines worldwide are refitted according to the invention and used as digestion plants, carbon dioxide emissions of more than 150,000 t/year can be avoided.

### Method and Plant for Producing Biogas from Biomass

### LIST OF LABELLINGS

1 Mixing and stacking vessel

2 Opening

3 Stirring device

4 Level

5 Block heat and power plant

6 Level

7 Drift

8 Drilling

9 Gas collection point

10 Blind shaft

11 First and second ventilation shaft

12 Third ventilation shaft

13 Closed ventilation shaft

14 Cover

15 Surface

1. Plant for producing biogas from different biomasses from households, agriculture, forestry, industry and commerce sectors (bio-organic residues) by anaerobic alkaline sludge digestion by means of different strains of methane bacteria, with a digester and a feed pipe for the bio-organic residues, wherein the digester consists of at least two subterranean, roughly horizontal cavities in a disused hard coal mine, left over from earlier mining activities, and wherein all cavities of the mine are interconnected at one or several points by defined lead-off drillings in such a way that these drillings all open to a gas collection reservoir situated at the highest point of the mine.

2. Plant for producing biogas according to claims 1, wherein these drillings measure approximately 20 cm in diameter.

3. Plant for producing biogas according to claim 1, wherein the gas collection reservoir has extraction facilities and connection pipes in order to transport the gas to linked-up utilisation facilities for power generation.

4. Plant for producing biogas according to claim 1, wherein an entrance shaft of the disused hard coal mine is intended for the introduction of the bio-organic residues.

5. Plant for producing biogas according to claim 1, wherein the upper 5 to 10 m of the entrance shaft are divided off as mixing and stacking vessel for receiving the bio-organic residues, the vessel being equipped with a stirring device, a cover for heat insulation as well as a pneumatically driven slide at the bottom end for discontinuous opening and the introduction of the bio-organic residues.

6. Plant for producing biogas according to claim 1, wherein the gas collection reservoir is connected via feed pipes to a gasometer for intermediate storage of gas accumulated in high quantities and not immediately usable.

7. Plant for producing biogas according to claim 1, wherein the temperature inside the digester is maintained in a range of 5° C. to 70° C. only by means of the geothermal power and by self-heating during the digestion process.

8. Plant for producing biogas according to claim 1, wherein all shafts and drifts not intended for the extraction of gas or the introduction of the bio-organic residues are hermetically closed and sealed at the surface.

9. Plant for producing biogas according to claim 8, wherein before the introduction of the bio-organic residues into the mine, the digester is charged at normal pressure with a mixture of air and at least one of the following components: natural gas, biogas and propane-butane mixture.

10. Method for producing biogas from biomasses, in which method different organic waste materials from households, agriculture, forestry, industry, and commerce sectors are introduced into at least one naturally existing digester and converted into methane containing biogas according to the principle of anaerobic alkaline sludge digestion by means of different strains of methane bacteria, wherein the bio-organic residues are introduced in great quantities and without prior degradation into at least two subterranean cavities in a disused hard coal mine, left over from earlier mining activities and serving as digester, interconnected by drillings, and wherein the sludge digestion is carried out in long-term reactions at a naturally existing temperature level of 5° C. to 70° C.

11. Method for producing biogas according to claim 10, wherein the cavities are interconnected at one or several points by defined lead-off drillings in such a way that these drillings all open to a gas collection reservoir situated at the highest point of the mine.

12. Method for producing biogas according to claim 11, wherein the biogas is transported from the gas collection reservoir via extraction facilities and connection pipes to the utilisation facilities for power generation which are linked up as required.

13. Method for producing biogas according to claim 10, wherein the bio-organic residues are introduced into a hard coal mine already refitted for mine gas production and utilisation, via at least one drilling towards the lowest level, into cavities and in that the biogas produced here is mixed with still produced mine gas and extracted for subsequent utilisation.

14. Method for producing biogas according to claim 10, wherein the temperatures in the range of 5° C. to 70° C., which are necessary for the living and reaction conditions of the methane bacteria in the digester, are maintained by means of the geothermal power existing in the mine in connection with the self-heating due to the anaerobic biological decomposition of the bio-organic substances, and wherein the conversion of the bio-organic residues is carried out in the cryophilic, mesophilic and/or thermophilic temperature range as well as in intermediate ranges such as the mild range.

15. Method for producing biogas according to claim 10, wherein the bio-organic residues are charged with seed sludge during the introduction process and in that the thus seeded bio-organic residues are mixed with digestion sludge already contained in the digester by pressing in natural and/or biogas.

16. Method for producing biogas according to claim 10, wherein before the introduction of the bio-organic residues in the mine, all shafts and drifts which are not intended for the extraction of gas or the introduction of bio-organic residues are closed and sealed up hermetically at the surface, then a negative pressure is produced by sucking out the existing air and immediately afterwards, natural gas, biogas and/or a propane-butane mixture is introduced until pressure balance is achieved.
